(19) United States
(12) Patent Application Publication
(10) Pub. No.: US 2025/0012684 A1
(43) Pub. Date: Jan. 9, 2025

(54) DEVICE FOR ISOLATION OF CELLS FROM URINE SAMPLES

(71) Applicant: ITAYANDBIOND LTD, Tel Aviv-Yaffo (IL)

(72) Inventors: Shmulik BEZALEL, Pyrgos, Limassol (CY); Jonatan DARR, Holon (IL); Nisim PERETS, Tel Aviv (IL); Yair TAMIR, Tel Aviv (IL)

(21) Appl. No.: 18/696,692

(22) PCT Filed: Sep. 29, 2022
(86) PCT No.: PCT/IL2022/051039

§ 371 Date: Mar. 28, 2024

Related U.S. Application Data
(60) Provisional application No. 63/250,265, filed on Sep. 30, 2021.

(51) Int. Cl. G01N 1/40; C12M 1/00

(52) U.S. Cl. CPC G01N 1/4077; C12M 47/04; G01N 2001/4088

(57) ABSTRACT

A device and a method are disclosed for a selective isolation and/or growing of matters of interest from a biological source fluid sample. A first part of the device includes a first compartment configured to collect the fluid sample, and a second compartment comprising a growth solution configured to preserve and/or facilitate growth of the matters of interest. A second part of the device comprises a third compartment, configured to capture the matters of interest by one or more filters. Further, the first compartment comprises a one-way valve which when open allows flow of the fluid sample from the first compartment into the third compartment through the one or more filters. Then, flow of the growth solution from the second compartment into the third compartment occurs, such that the matters of interest captured by the one or more filters are covered by the growth solution.

### TECHNICAL FIELD

The present disclosure relates generally to a device and a method for selective isolation and/or growth of matters of interest from a fluid sample.

### BACKGROUND

Fluid samples originating either from a biological source, such a human or an animal, or from an inanimate source such as seas, often include various types of matters of interest, such as cells, peptides, viruses, and a wide variety of different organic and inorganic substances. Analyzing a fluid sample provides an insightful information regarding the fluid sample source, presence of contaminations and infections. Analyzing a fluid sample from a biological source is of critical importance for understanding not only the properties of the fluid sample, but also the condition of the subject, the diagnosis and the optional treatments techniques. Although several types of isolating of the matters on interest exist, they typically require laboratory facilities, such as chromatography, and well-trained personnel for performing the analysis as well as suitable growth conditions. Although very accurate techniques were developed for identifying presence of matters, with a detection limit of up to several cells, these are generally expensive, require long isolating times and sophisticated equipment, which, in turn, limits their implementation in identification of matters of interest with a short shelf life, and may be not applicable in rural areas or for field applications.

Thus, there is a need in the art for a mobile and technically simple device enabling fast and selective isolation and preservation of matters of interest from a fluid sample.

### SUMMARY

Aspects of the disclosure, according to some embodiments thereof, relate to a device and a method for a selective isolating of matters of interest from a biological source sample that can be performed in a laboratory or by a point-of-care user in any location by a technically simple method and without the need for sophisticated equipment.

According to some embodiments, the disclosed device comprises two parts, a first and a second part, and a plurality of compartments, the first part includes a first compartment utilized for collecting the fluid sample, and a second compartment, which includes a growth solution used to preserve and/or to facilitate growth of the matters of interest. The second part includes a third compartment, comprising one or more filters for capturing the matters of interest. The first compartment comprises a one-way valve that governs the fluid sample flow. In an open configuration, the one-way valve allows flow of the fluid sample of the first compartment into the third compartment through the one or more filters. Then, e.g., due to the applying of pressure on the device, growth solution flow is initiated from the second compartment into the third compartment, covering the captured matters of interest by the growth solution.

Optionally, the first part may include a fourth compartment, configured to retain washing solution, allowing washing out of the fluid sample after passing through one or more filters. The second part may optionally include a fifth compartment, configured to accumulate and store waste and/or fluids, such as remaining portion of the fluid sample (i.e., after filtering) and/or washing solution.

The herein disclosed device advantageously provides a technically simple, mobile, fast, and a point-of-care device and method that can mitigate the time constrains and reduce the labor currently required, especially, but not exclusively, in performing diagnostic tests and in other uses such as personalized medicine.

Furthermore, the device is configured to allow sustained viability, at least for a duration of hours (e.g. 2 hours, five hours or more), and even growth of the collected matter of interest. This may be of particular importance when further expansion and or manipulation of the matter of interest is required,

As a non-limiting example, the device may be used for capturing epithelial cells from a urine sample obtained from a subject. Since the cells are provided with essential requirements for maintaining their viability through the growth solution, the cells can subsequently be released from the device and be used for further analysis and manipulation, including transforming the cells into Induced Pluripotent Stem Cells (IPSCs). Advantageously, patient derived IPSCs can enable personalized drug screening and development. Furthermore, IPSC can be differentiated into other cell types, for example neurons, or hematopoietic stem cells, which can be further used in newly developed clinical treatments and assist in the prediction of clinical effects. Another, non-limiting example, the device may be use for bladder, prostate or other urinary secreted cancer cells diagnosis by capturing living cancer cells and expanding them in the lab for diagnosis, drug screening and personalized drug development.

One of the major limiting factors for performing advanced analysis in rural areas and/or by untrained end-users is the short viability of some of the isolated matters of interest. Further, subjects with neurological disorders, including mental and physical disabilities, such as autism spectrum disorder (ASD), may be unpredictable, uncooperative, and challenging to bring to a medical facility, thereby requiring not only a mobile but also a fast diagnostic device.

Moreover, the device and method disclosed herein, according to some embodiments, adventitiously allow selective isolation of matters of interest (including exosomes, extracellular vesicles, apoptotic bodies, cell-free RNA or DNA, etc.) within a short time, maintaining the viability, integrity as well as growth, expansion, and culturing thereof. This may for example be of in remote environments which may require prolonged storage prior to being collected.

In addition, in some embodiments, selective isolation and/or growth device may allow manipulation of matters of interest (such as cells and exosomes), e.g., re-purposing of exosomes (e.g., loading the exosomes with active substances and/or manipulating their targeting capabilities) and/or epithelial cells isolation and their transformation into various cell linages from IPSCs.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1A shows a schematic illustration of a cross-sectional side view of first and second parts of selectively isolating and/or growing device, according to some embodiments;

FIG. 1B shows a schematic illustration of a cross-sectional side view of first and second parts of selectively isolating and/or growing device after association, according to some embodiments;

FIG. 2A shows a schematic illustration of a side view of selectively isolating and/or growing device, according to some embodiments;

FIG. 2B shows a schematic illustration of a top perspective view of selectively isolating and/or growing device of FIG. 2A, according to some embodiments;

FIG. 3A shows a schematic illustration of a perspective view of a second part of selectively isolating and/or growing device, according to some embodiments;

FIG. 3B shows a schematic illustration of a perspective view of one or more filters of third compartment shown in FIG. 3A, according to some embodiments;

FIG. 3C shows a schematic illustration of top and side views of a filtering layer positioned in of one or more filters, according to some embodiments;

FIG. 4 shows a flow chart of a selective isolation and/or growth method of matters of interest, according to some embodiments;

FIG. 5A shows a light microscope image of a fluid sample comprising bladder cancer cells before loading into a first experimental device, fabricated according to some of the embodiments;

FIG. 5B shows a light microscope image of a post-filtered fluid collected after passing through the first experimental device, fabricated according to some of the embodiments;

FIG. 6A and FIG. 6B show light microscope images of bladder cancer cells captured in filters of the first experimental device, fabricated according to some embodiments;

FIG. 7 shows the capacity of a filtering layer of the present disclose compared to the capacity of a control filtering layer, according to some embodiments;

FIG. 8 shows live and healthy urine cells captured/trapped in a filtering layer, according to some embodiments;

FIG. 9 shows live and healthy urine epithelial cells captured/trapped in the filtering layer following counting their respective counting and dilution to 3000 cells per 100 ml urine and staining thereof with a red cytopainter and a blue nuclear staining, according to some embodiments;

FIG. 10 shows the compatibility for capturing/trapping of additional cell types, such as cells for cancer diagnosis, according to some embodiments;

FIG. 11 shows indications of size-selective separation of the disclosed herein device, according to some embodiments;

FIG. 12 shows a flow simulation plot depicting liquid with cells flowing from left to right though a disclosed herein device, according to some embodiments; and

FIG. 13 shows an example of a trapping grid of one or more filters, according to some embodiments.

### DETAILED DESCRIPTION

The present invention, in some embodiments thereof, relates to medical devices, and, more specifically, to a method and a device for a selective isolation and/or growth of matters of interest from a fluid sample.

In the description and claims of the application, the words “include” and “have”, and forms thereof, are not limited to members in a list with which the words may be associated.

The term “fluid sample” as used herein refers to any fluid sample wherein the components of the fluid can be isolated. The fluid sample may be an untreated sample, i.e., as received and/or directly collected into the device, or a treated sample, i.e., following a thermal treatment and/or a chemical treatment, such as dilution of the fluid sample, adding and/or mixing with a solution, a buffer, or any material, to facilitate the selective isolation, preservation and/or growth processes.

As used herein, a “source” refers to any source of a fluid sample, such as a biological source, including a human source or an animal source, such as mammals and non-mammals, and/or an inanimate source, such as rivers, seas, lakes, waterfalls, ponds, and the like.

The term “matters of interest” as used herein refers to any particles, molecules, cells, and materials structures of any shape and composition that can by isolated and/or grown from a fluid sample by physical and/or chemical and/or electronic interactions, and/or by utilizing filters. For instance, matters of interest may include cells, such as epithelial cells, organelles such as exosomes, extracellular vesicles, apoptotic bodies, cell-free RNA or DNA, proteins, peptides, hormones, viruses, bacteria, crystals, pollutants, and the like. Each possibility is a separate embodiment. Matters of interest may be soluble or insoluble in the fluid sample. The typical size of the matters of interest is in the micrometer and nanometer scales dimensions.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 mm” is equivalent to the statement “the length of the element is between 0.8 mm and 1.2 mm”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “approximate” and “about” may be interchangeable.

As used herein, the term “filter” refers to any type of filtering and/or fractionation element or a combination of filters/fractionation elements for selectively isolating, preservation and/or growing of matters of interest from a fluid sample, including microporous and/or nanoporous filters, membranes filters, meshes, grids, microscopic and/or nanoscopic ridges, gravity filters, vacuum filters, multi-layer filters, cell strainers, chip-based filters including at least one pore, dielectrophoretic antibodies, filter papers, traps, such as cell traps, microfluidic systems, such as inertial microfluidics, and the like. Each possibility is a separate embodiment.

According to some embodiments, filter may include a device utilizing inertial microfluids for a selective separation of matters of interest based on their size and/or weight. In contrast to several known separation techniques, such as dielectrophoresis and optical tweezers, inertial microfluidics selective isolation does not require application of external forces, and depend only on the hydrodynamic properties of the fluid and channels geometry. This is of uttermost importance when continuous viability and growth of the captured material is desired.

According to some embodiments, filter may include a device utilizing micro and/or nano-filters in various sizes for size-based separation of matters of interest. According to some embodiments, the size-specific separation may be achieved by decreasing the diameter of the pore of the nanofilter along its length. According to some embodiments, the size-specific separation may be achieved by utilizing ridges of various sizes and/or shapes.

According to some embodiments, filter may include one or more devices, wherein each of the devices applies different separation mechanism, for example, combining weight and magnetic separation.

Optionally, filters may include surfactants, micelles, or any other molecules and/or organic and/or inorganic materials that bind and/or capture and/or preserve certain materials, either to the matters of interest or to the undesired components of the sample, to facilitate the selective isolation and/or preservation and/or growth of the matters of interest.

Filters may isolate the matters of interest according to the difference in their size, weight, electrostatic forces, electromagnetic forces, affinity, conductivity, ion exchange procedures, and/or any other procedure (or a combination of thereof) allowing separation of the matters of interest. Each possibility is a separate embodiment. Further, filters may be manufactured from ceramics, metals, polymeric or organic materials, such as nylon, polycarbonate, cellulose-based materials, polyether sulfone, silicon wafers, and the like. Each possibility is a separate embodiment. Optionally, filters may include wetting solution for enhancing fluid sample flow.

According to some embodiments, such as inertial microfluids separation, the filters may include channels, such as micro and/or nano-channels in various geometries and sizes. In some embodiments, the geometry of the channels may be straight, triangular, curved, spiral, serpentine, or a combination of thereof, to facilitate modifying of the equilibrium positions of matters of interest. Further, the channel geometry may include disturbance structures, such as micro-pillar obstacles, contraction-expansion cavities, for tailoring fluid flow properties.

According to some embodiments, a device for selectively isolating and/or growing of matters of interest may be a 3D-printed device. According to some embodiments, the device may be 3D-printed by a stereolithography (SLA) printing technique. According to some embodiments, the device may be 3D-printed from biocompatible resins.

According to some embodiments, the resins may include polymer-based resins. According to some embodiments, the resins may include ultimate tensile strength (UTS) values in the range of about 50-75 MPa as measured by ASTM D638-10 (Type IV). According to some embodiments, the resins may have post-curing elongation of about 11-13% as measured by ASTM D638-10 (Type IV). According to some embodiments, the resins may have a minimal flexural strength of about 75 MPa as measured by ASTM D790-15 (method B). According to some embodiments, the resins may have shore D hardness values of about 60-80 D as measured by ASTM D2240-15 (Type D). According to some embodiments, the resins may be compatible with chemical disinfection by alcohols, such as 70% isopropyl alcohol.

As non-limiting examples, the resins may include BioMed Clear, BioMed Amber resins by Formlabs, surgical guide resin and/or dental LT clear (V2) by Formlabs.

According to some embodiments, post-printing washing of the device or of one or more compartments of the device may be required. According to some embodiments, post-printing washing may include two stages, the first stage may include washing of the device in alcohol, such as isopropanol 95%, for removing an uncured resin, and the second stage may include a subsequent washing in >99% isopropanol for removing the remnants of the uncured resin, followed by thorough drying of the device (e.g. by compressed air or nitrogen flow).

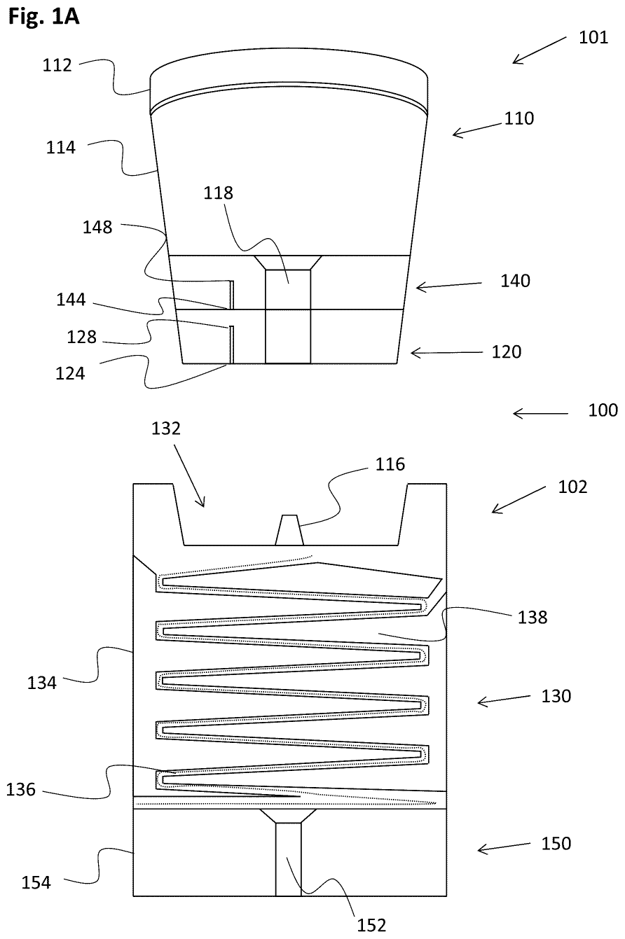

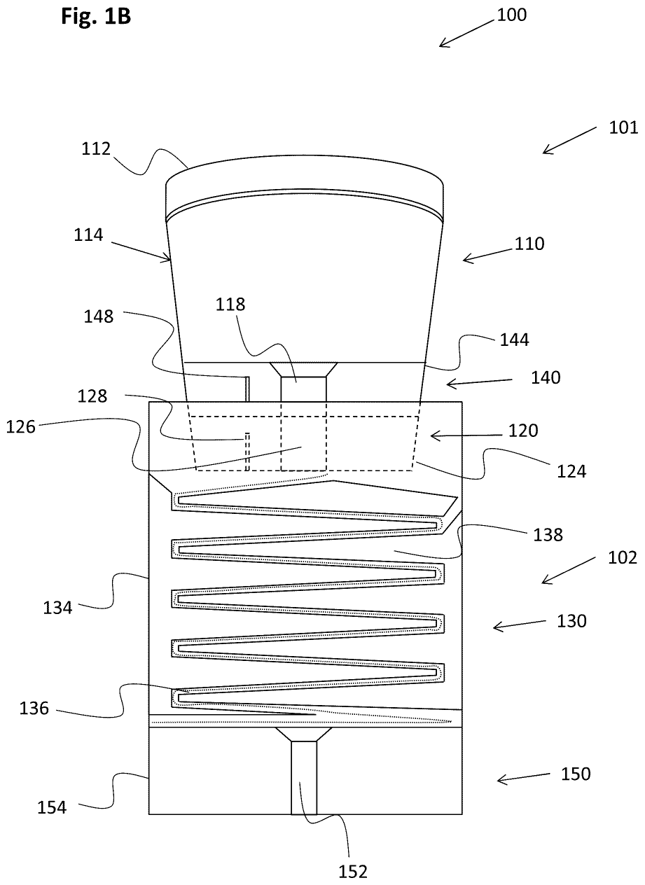

According to some embodiments, the device is compatible with various biological sterilization methods, including autoclave, gamma rays, electron beam (e-beam), and ethylene oxide (EtO) sterilizations. Each possibly is a separate embodiment. Reference is now made to FIG. 1A and FIG. 1B, which schematically illustrate a cross-sectional sideview of a device 100 for selectively isolating and/or growing a matter of interest, according to some embodiments. Device 100 may include two parts: A first part 101 is a fluid collection part configured to receive and/or store samples including a matter of interest, as well as to contain various liquids utilized for the isolation and/or growth of the matter of interest. And a second part 102 which is a capturing part configured to capture matter of interest from the received sample. First part 101 preferably includes a plurality of compartments: a first compartment 110 configured to receive and optionally store fluid sample (e.g. a urine sample) containing matters of interest (e.g. epithelial cells and/or exosomes), a second compartment 120 which may contain a washing solution, and a fourth compartment 140 configured to contain a growth solution configured to ensure the viability and/or growth of the matter of interest. Second part 102 preferably includes a plurality of compartments: a third compartment 130 configured to isolate optionally to preserve and/or grow matters of interest, and a fifth compartment 150 configured to receive fluids after passing through third compartment 130. The received fluids may include post-filtered fluid sample (i.e., a remaining portion of the fluid sample after isolating matters of interest), and/or washing solution

According to some embodiments, fifth compartment 150 may include one or more sub-sections (not shown), configured to separate post-filtered fluids and waste. The sub-sections of the fifth compartment 150 may include a first sub-section for accumulating and retaining the post-filtered fluid sample, and a second sub-section for accumulating and retaining the washing solution after passing through third compartment 130.

FIG. 1A shows first part 101, including first, second and fourth compartments, 110, 120 and 140, prior to associating with second part 102, including third and fifth compartments, 130 and 150, according to some embodiments.

As shown in FIG. 1A, device 100 further includes a one-way valve 116, which may be positioned in a base 132 of second part 102, as depicted in FIG. 1A. One-way valve 116 possesses two configurations, an open configuration, allowing fluid sample flow from first compartment 110 to third compartment 130 through conduit 118, and a closed configuration, retaining fluid sample inside container 114, i.e., preventing fluid sample flow into third compartment 130. The configuration of one-way valve 116 may be governed by various mechanisms, e.g., mechanical pressure, vacuum, and the like, as further elaborated herein. According to some embodiments, one-way valve 116 may include Bernoulli and/or pressure pump, configured to control fluid sample flow for initializing the selective isolating and/or growing.

According to some embodiments, first compartment 110 may include a cap or lid 112, such as a silicon lid, configured to associate with container 114 (i.e., a cup, a beaker) for securing fluid sample inside device 100. Optionally, first compartment 110 may include one or more puncturing needles (not shown). One or more puncturing needles may be implemented for initiating flow of the fluid sample from first compartment 110 into third compartment 130.

While other mechanisms may also be envisaged, as elaborated herein, second compartment 120 and fourth compartment 140 may include puncturing needles, such as needles 128 and 148, respectively, configured to puncture the barriers 124 and 144 fluidly separating second compartment 120 and fourth compartment 140 from first compartment 110. This may advantageously allow a timed (i.e., sequential) flow from each of compartments 110, 120 and 140 through one-way valve 116 and into compartment 130 containing one or more filters 138.

For example, initially fluid flow of the fluid sample contained in first compartment 110 may flow through one-way valve 116 into third compartment 130, for example as a result of pressure being manually applied on cap 112. In third compartment 130 the matters of interest of the fluid sample may be captured by one or more filters 138. Thereafter, for example as a result of activation of needle 128 flow of liquid (e.g. saline or other washing media) may be facilitated from second compartment 120 into third compartment 130 through barrier 124, thereby washing the matter of interest (e.g. removing residual urine). Next, due to activation of needle 148 flow of liquid (e.g. growth media) may be allowed from compartment 140 into compartment 130, thereby supplying the matter of interest with the required conditions for its continued viability and/or growth.

According to an alternative embodiment, one way valve 116 may open automatically as a result of association of first part 101 with second part 102 (without requiring manual pressure to be applied). As yet another alternative, one way valve 116 may open as a result of filling a sufficient volume (e.g., at least half capacity of compartment 110 or any other minimum volume). Third compartment 130 includes base 132, configured to receive and secure first part 101 to second part 102. Third compartment 130 further includes a channel 136 configured to allow passing of fluids (schematically denoted by a dotted line in FIG. 1A and FIG. 1B) through third compartment 130. According to some embodiments, third compartment 130 may include one or more filters 138 configured to capture the matters of interest. According to some embodiments, capturing the matters of interest occurs during flowing of the fluid sample (such as a urine sample) through channel 136 and/or one or more filters 138. Matters of interest are selectively isolated according to the difference in their physical and/or chemical and/or electric properties and/or a combination of the properties thereof with respect to other components in the fluid sample. Each possibility is a separate embodiment.

According to some embodiments, height of channel 136 may facilitate laminar fluids flow. In some embodiments, channel 136 height may be within the range of about 0.5-2 mm or 0.75-1.5 mm.

One or more filters 138 may include membranes, microporous and/or nanoporous filters, inertial microfluidics system, microscopic and/or nanoscopic ridges, channels, and the like. Each possibility is a separate embodiment. In some embodiments, one or more filters 138 may include filters with various properties, such as filters with different sizes for size-based separation of matters of interest, and/or filters combining different separation mechanisms, such as size-based and electron affinity-based separations.

According to some embodiments, the one or more 138 filters may include ridges-based filters. According to some embodiments, the dimensions (e.g. height, width, and length) of ridges in each of the one or more filters 138 are essentially the same. According to some embodiments, the dimensions of the ridges in each of the ridges-based filters may vary.

According to some embodiments, the ridges height may be in the range of about 50-500 μm. According to some embodiments, ridges width may be within the range of about 600-1000 μm. According to some embodiments, ridges length may in the range of about 600-1800 μm.

According to some embodiments, one or more filters 138 may include three filters. As a non-limiting example, the three filters may include ridges-based filters with the same dimensions. As a non-limiting example, the three filters may include ridges with the following approximate dimensions: height 300 μm, length 1 mm, and width 826 μm. In another non-limiting example, the approximate dimensions of the ridges may include height of 250 μm, length of 1 mm, and width of 826 μm. In another non-limiting example, the approximate dimensions of the ridges may include height of 350 μm, length of 1 mm, and width of 826 μm. Alternatively, the three filters may include ridges-based filters with different dimensions. According to some embodiments, the ridges may be dimensioned and/or shaped to capture different matter of interest. As a non-limiting example, different sized/shaped ridges may capture cells of different sizes. As another non-limiting example, some ridges may be sized/shaped to capture cells, whereas other ridges may be sized/shaped to capture exosomes.

According to some embodiments, one or more filters 138 allow identifying presence and/or concentration of matters of interest in fluid sample. In some embodiments, third compartment 130 may be equipped with sensors and/or indicators, such as color-changing indicators, thereby providing qualitive and/or quantitative information related to content of the fluid sample and/or other fluids (i.e., washing solution, growth solution, etc.) present in device 100.

According to some embodiments, device 100 may include one or more detectors and/or sensors (not shown) for analyzing the filtered fluid sample. In some embodiments, the one or more detectors and/or sensors may monitor parameters, such as inertial microfluid-related parameters, to evaluate the quality performance of device 100. According to some embodiments, the one or more detectors and/or sensors may measure and/or collect data related to the matters of interest, including its concentration, viability, type, growth rate or any combination thereof. Each possibility is a separate embodiment.

Optionally, one or more detectors may be compatible with computer-based devices, including smartphones, computers, multimedia devices, and any hand-held programmable devices, configured to receive and process data collected by the one or more detectors. Then, end-user and/or medical personnel may conveniently receive the processed data related the fluid sample, matters of interest, and/or general state and function of device 100.

According to some embodiments, one or more filters 138 may be removably associated with compartment 130. Different scenarios wherein it may be desirable to remove one or more filters 138 from container 134 may occur, including analyzing of matters of interest (e.g., microscopic imaging), or when transferring matters of interest is required.

According to some embodiments, each of compartments in first part 101 (e.g., first, second, and fourth containers 110, 120, and 140) may include a one-way valve configured to allow automatic and timely configuration shift for initiating sequential flow of fluid sample, washing solution, and growth solution through third compartment 130. In some embodiments, each of one-way valves may be manually shifted. According to some embodiments, device 100 may include one or more external buttons for shifting the configuration of one-way valve 116, and optionally, of other one-way valves. According to some embodiments, device 100 may include one or more timers, wherein one or more timers may be internally and/or externally associated with each of the compartments in first part 101, and/or with third compartment 130, allowing measurement of the time duration required for filtering of fluid sample, and/or for providing growth solution and/or washing solution. Optionally, one or more timers may be communicatively associated with each of one-way valves for allowing timely configuration shift of each of one-way valves, thus providing precise flow initialization of washing solution, and/or growth solution, and/or post-filtered fluids.

According to some embodiments, each of compartments in first part 101 (e.g., first, second, and fourth containers 110, 120, and 140) may admit each of fluids (i.e., fluid sample, and/or washing solution, and/or growth solution) into third compartment 130 in several stages. For example, growth solution retained in fourth compartment 140 may be delivered into third compartment 130 by three stages (i.e., each delivery includes third of the initial volume of the growth solution) for providing sufficient amount of nutrients enhancing preservation and cells growth.

Growth solution allows selective preservation of matters of interest, such as epithelial cells or cancer stem cells, and any cells and/or materials with a short shelf life. Optionally, growth solution may preserve the viability of the isolated matters of interest by supplying the necessary preservation and/or growth conditions.

Optionally, second compartment 120 may include one or more puncturing needles 128. One or more puncturing needles 128 may implemented for flow initiating of the growth solution from second compartment 120 into third compartment 130.

According to some embodiments, first compartment 110 may be made of softer and/or more flexible and/or foldable materials than second and fourth compartments 120 and 140. Consequently, upon applying mechanical pressure on first compartment 110 (e.g., on cap 112), the first compartment 110 will fold first. Once the first compartment 110 is folded, second and third compartments 120 and 130 may start to fold. Folding of the second compartment 120 applies pressure on puncturing needle 128 (since it is longer than puncturing needle 148), thereby puncturing barrier 124 and allowing flow of washing solution from second compartment 120 to third compartment 130. Then, upon applying additional pressure, puncturing needle 148 will puncture barrier 144, thereby allowing flow of growth solution from fourth compartment 140 into third compartment 130 via second compartment 120.

According to some embodiments, fifth compartment 150 configured to receive via an outlet 152 waste such post-filtered fluids, including post-filtered fluid sample, and/or post-filtered washing solution. Fifth compartment may be permanently or detachably associated with second part 102.

Optionally, outlet 152 may include a one-way valve. According to some embodiments, outlet 152 may include timing and/or activation mechanism, which may allow the shifting of outlet 152 (e.g., one-way valve) from an open to a closed position after washing (e.g., closing the one-way valve after washing one or more filters 138 with washing solution). Put differently, shifting to closed configuration prior to initiating flow of the growth solution so as to maintain growth solution within third compartment 130 and cover the matters of interest captured by one or more filters 138. FIG. 1B shows first part 101 assembled with second part 102 via base 132.

As previously noted, flow of the fluid sample from first compartment 110 into third compartment 130 may be initiated by associating first part 101 with second part 102, optionally, with or without applying an external pressure.

According to some embodiments, second compartment 120 may be detachably associated or integrally formed with first compartment 110 and/or third compartment 130, for example, by base 132 as shown in FIG. 1A.

According to some embodiments, fourth container 140 may be removably or permanently associated with first compartment 100 and/or with second compartment 120 and/or with third compartment 130.

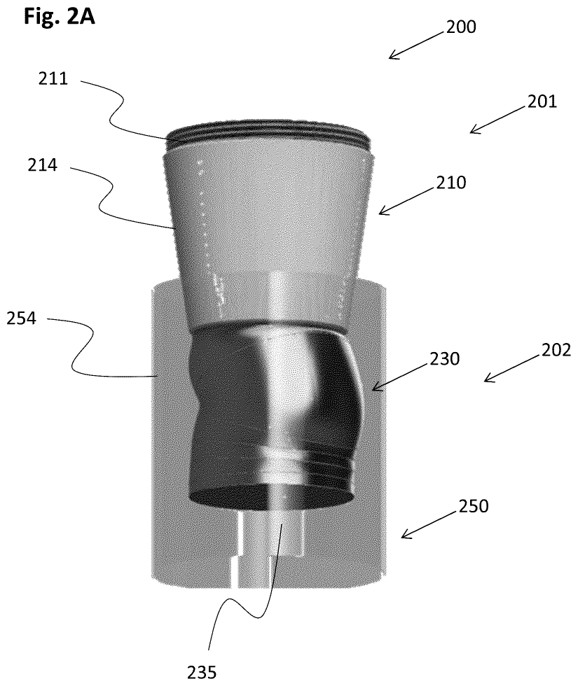

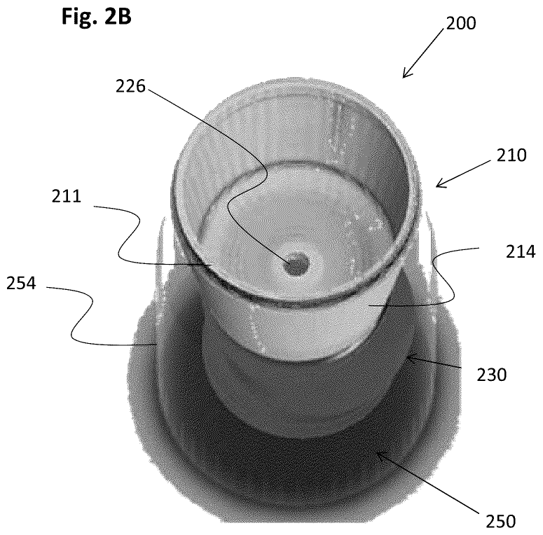

Reference is now made to FIG. 2A, which schematically illustrates a side view of device 200 for selective isolation and/or growing of matters of interest, according to some embodiments. Device 200 includes a first part 201 and a second part 202. First part 201 includes first compartment 210, configured to collect a fluid sample, while second and fourth compartment are concealed and located within first compartment 210. Second compartment (concealed) is configured to retain growth solution, and fourth compartment (concealed) is configured to retain washing solution. Second part 202 includes third compartment 230, configured to isolate, preserve, and enhance growth of matters of interest.

According to some embodiments, second part 202 may include fifth compartment 250 configured to collect waste. According to some embodiments, the fifth compartment may be configured to collect the remainder of the fluid sample having passed through third compartment 230 (also referred to herein as post-filtered fluids or post filtered sample)

According to some embodiments, each of the disclosed devices, such as device 100 and 200, may include a case 254, such as an electronic case, configured to record and/or transfer data regarding matters of interest and/or performance of device 200. Optionally, data recorded by case 254 may include temperate measurements, humidity measurements, angular velocity and/or orientation, concentration of matters of interest, and the like. In some embodiments, the casing may provide thermal insulation or allow for a controlled thermal environment keeping the fluid in a desired temperature. In some embodiments, collection of the data may be performed in each or some of the compartments (i.e., first, second, third, fourth and/or fifth compartment) and transferred to case 254. Optionally, the recorded data may be further transferred to electronic devices, such as smartphones, and to medical and/or scientific staff.

According to some embodiments, first compartment 210 includes a container 214 (e.g., a cup or a beaker), and may include a thread 211 configured to associate with a cap or a lid (not shown), for retaining fluid sample inside first compartment 210, and preventing undesired fluid sample loss.

Third compartment 230 includes one or more filters (concealed), and optionally may include an output 235 for draining waste from third compartment 230 into fifth compartment 250. In some embodiments, waste draining via output 235 may occur automatically, including flow generated by gravity, pump, valve, and the like, or manually, such as by a syringe. In some embodiments, output 235 may include a one-way valve, allowing draining of fluids from third compartment 230 at predefined points of time and/or time durations. For example, in some embodiments, it might be beneficial to retain growth solution in one or more filters for a predefined time duration for facilitating growth and/or preservation and/or enabling manipulation of matters of interest.

In some embodiments, third compartment 230 may include channels (not shown) for facilitating selective isolation of matters of interest from fluid sample, and enhancing separation of cells and/or other substances therefrom. According to some embodiments, channels may refer to microchannels and/or nanochannels configured to alleviate clogging and/or facilitate laminar fluid flow. In some embodiments, channels may be utilized for selective isolation of matters of interest, such as in inertial microfluidic separation method. Optionally, third compartment 230 may include one or more filters and/or channels for differential filtration technique, such as size-based isolation, thereby capturing larger substances than the matters of interest, while allowing passage of substances smaller or similar to the size of matters of interest.

Reference is now made to FIG. 2B, which schematically illustrates a top perspective view of device 200, according to some embodiments. One-way valve 226 may be positioned at the bottom of first compartment 210, for regulating and initiating fluid sample flow by various mechanisms, such as pressure. For example, one-way valve 226 may be a pressure valve configured to open when external pressure is applied, e.g., manually on a lid (not shown) closing of first compartment 210. As another example one-way valve 226 may include a sealing flap and/or a shutter, which shifts to an open configuration by applying pressure upon associating first compartment 210 with third compartment 230, thereby initiating fluid sample flow. In other embodiments, one-way valve 226 initializes fluid sample flow when first compartment 210 is substantially full (i.e., at least half of the volume is filled).

According to some embodiments, container 214 may include a cup with a collapsible configuration (not shown) for biological source sample collection. Optionally, cup with a collapsible configuration may include a slide line to facilitate folding and unfolding thereof. This may for example advantageously allow the folding of container 214 when pressure is applied on the lid attached thereto, thereby increasing the pressure within container 214 and causing the opening of one-way valve 226.

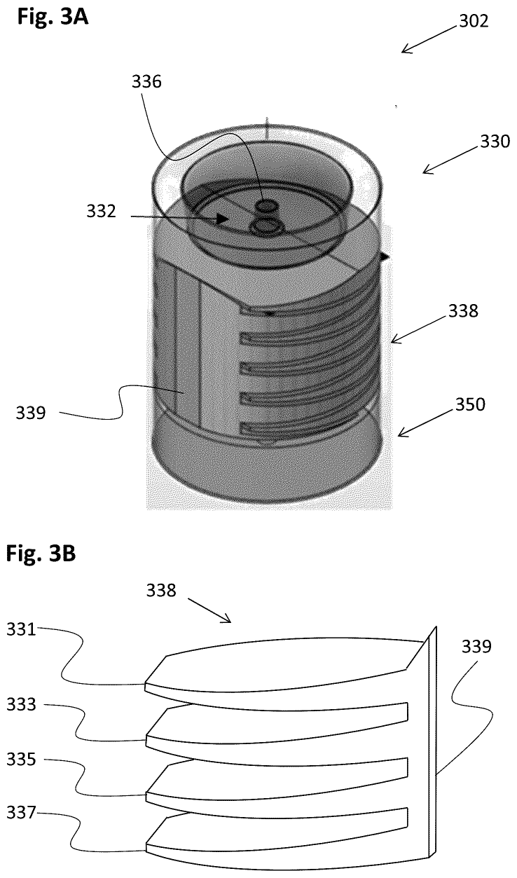

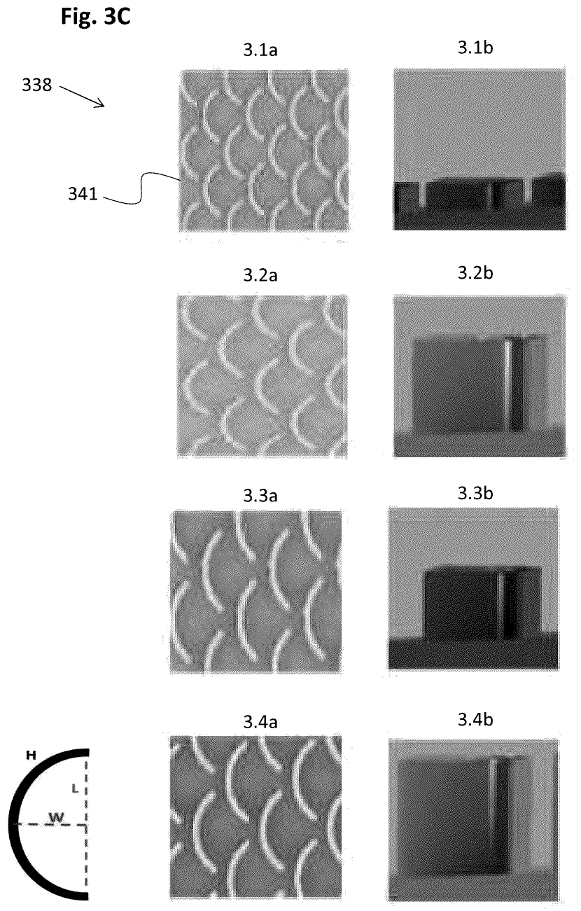

Reference is now made to FIG. 3A, which schematically illustrates a perspective view of a second part 302, according to some embodiments. Second part includes a third compartment 330, which includes a base 332, configured to hold and secure first part (not shown) upon association with second part 302, and a one-way valve 336 configured to govern the fluid sample flow from first compartment (not shown) into third compartment 330. Third compartment 330 further includes one or more filters 338, configured to isolate, preserve, and enable growth of matters of interest from a fluid sample.

According to some embodiments, base 332 may exhibit various geometries (e.g., circular as depicted in FIG. 3A, rectangular, and the like) for retaining first part (not shown) of selective isolating and/or growing device. In other embodiments, first part may be permanently associated with second part 302 of the device.

According to some embodiments, third compartment 330 may include a holder 339 configured to hold and secure one or more filters 338. In some embodiments, holder 339 may be permanently or removably associated with third compartment 330. Optionally, holder 339 may be permanently or removably associated with one or more filters 338.

According to some embodiments, second part 302 may optionally include a fifth compartment 350 configured for waste collection.

Reference is now made to FIG. 3B, which schematically illustrates a perspective view of one or more filters 338 and a holder 339. As shown in FIG. 3B, one or more filters FIG. 3B may include four filtering layers 331, 333, 335, and 337. In other embodiments, the amount and the type of filtering layers may vary. According to some embodiments, filtering layers 331, 333, 335, and 337 may be permanently or removably associated with holder 339. Filtering layers 331, 333, 335, and 337 may be planar, optionally parallel, curved, and/or positioned at different angels and/or directions relatively to each other, for facilitating selective isolation of matters of interest and/or growth. According to some embodiments, filtering layers 331, 333, 335, and 337 may be angled and/or tilted to ensure a continuous flow through one or more filters 338 (or through channel 136). According to some embodiments, filtering layers 331, 333, 335, and 337 may be angled to ensure a predetermined flow rate through one or more filters 338 (or through channel 136). According to some embodiments, filtering layers 331, 333, 335, and 337 may be tilted so as to ensure flow therethrough. According to some embodiments, filtering layers 331, 333, 335, and 337 may be tilted by about 2-10 degrees, 3-7 degrees, 4-6 degrees or any other tilt in the range of about 1-15. Each possibility is a separate embodiment. According to some embodiments, the tilt may be in the range of about 1%-8%, 2%-6% or 2%-4% or any other suitable tilt within the range of 1%-10%. Each possibility is a separate embodiment. As a non-limiting example, filtering layers 331, 333, 335, and 337 may be tilted by about 5.4 degrees (a tilt of about 3%). In some embodiments, the tilt angle may be higher than about 3%. According to some embodiments, the tilt angle may be lower than about 3%. According to some embodiments, each of the filtering layers 331, 333, 335, and 337 may be tilted by a different angle. Each of the possibilities is a separate embodiment.

According to some embodiments, holder 339 may be removably associated with third compartment 330 and/or one or more filters 338. Optionally, holder 339 allows selective removing and/or inserting of each of filtering layers 331, 333, 335, and 337. Different scenarios wherein it may be desirable to remove each of filtering layers 331, 333, 335, and 337 may occur, including analyzing of matters of interest (e.g., microscopic imaging), or when transferring matters of interest is required.

According to some embodiments, selective isolating and fractionating of matters of interest may be based on inertial microfluids for separating matters of inters (or other matters, as an initial process step) according on their size and/or weight and/or other characteristics. This may be attained by a series of microscopic ridges, such as filtering layers 331, 333, 335, and 337, configured to capture matters of interest. For example, first and second filtering layers 331 and 333 may include micropores and/or micro ridges, configured to capture substances with larger size/weight than the matters of interest thereof, i.e., allowing passing of matters of interest (and optionally of smaller substances) toward next filtering system/s. Third and fourth filtering layers 335 and 337 may include nanopores and/or nano ridges, for preventing exiting matters of interest from one or more filters 338, and optionally, allowing transferring of smaller substances to exit third compartment 330 as waste.

According to some embodiments, selective isolating and fractionating of matters of interest may include micro and nano filters in variable sizes for size separating of thereof. Optionally, isolating of matters of interest may be performed based on other properties, such as electrostatic forces, electromagnetic forces, affinity, conductivity, ion exchange procedures, and/or any other procedure (or a combination of thereof).

According to some embodiments, one or more filtering layers 331, 333, 335, and 337 may be coupled with a heating element, including a hot plate, infrared heating source, and the like. In some embodiments, heating of isolated matters of interest may facilitate manipulation of matters of interest, and/or increase nucleation and growth rate, and/or enhance reactions rate, such as reactions involving presence of one or more precursors, and/or allow technically simple performance of curing or other thermal treatments to facilitate the isolating process, such as by dissociating thermally unstable substances. Each of possibilities is a separate embodiment.

Reference is now made to FIG. 3C, which schematically illustrates top and side views of filtering layer 341, positioned in one or more filters 338, according to some embodiments. According to some embodiments, filtering layer 341 may include ridges, such as microscopic and/or nanoscopic ridges, for selective isolation of matters of interest. Filtering layer 341 may include symmetric and/or asymmetric pores and/or ridges. Optionally, filtering layer 341 may include ridges with various sizes (e.g., with various values of length (marked as “L”) and width (marked as “W”) as depicted in the top views of FIGS. 3.1a-3.4a, and with various values of height (marked as “H”) as depicted in the side views of FIGS. 3.1b-3.4b), allowing isolating of larger species from fluid sample while smaller species continue to flow through next filtering layers, or through other separating elements. As a non-limiting example of the present disclosure, one or more filters 338 may include combined filtering system, such that (i) first, large substances (i.e., substances larger than matters of interest) are isolated using filtering layer 341, by size and/or weight, allowing passage of smaller substances (i.e., substances with equal or smaller size and/or weight than matters of interest) into next filtering system, and then, (ii) smaller substances are selectively isolated according to their electronic affinity. In some embodiments, one or more filters 338 may include various types of filtering layers 341, allowing isolating matters of interest (or a plurality of matters of interest) according to several mechanisms and/or isolating steps.

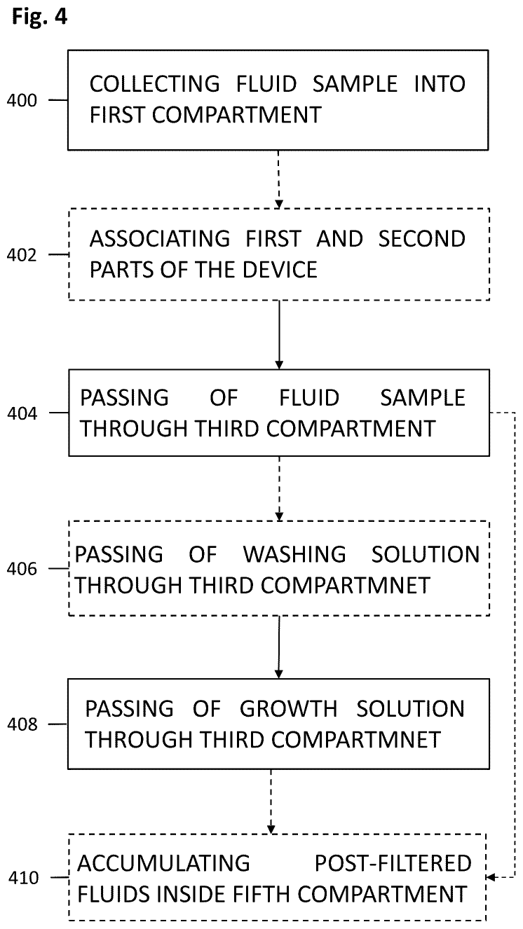

Reference is now made to FIG. 4, which shows a flowchart diagram of a method for selective isolation and/or growth of matters of interest, according to some embodiments.

In step 400, end-user collects a fluid sample into a first compartment of selective isolation and/or growth device. In some embodiments, fluid sample may originate from a biological source, such as human source (e.g. urine), and specifically, but not exclusively, subjects with neurological disorders, mental and/or physical disabilities, and/or cancer.

In step 402, which is an optional step according to some embodiments, the end-user may associate first and second parts of the device for selective isolation and/or growth of matters of interest. In some embodiments, associating the first and the second parts leads to one-way valve configuration shifting from closed to open position. Alternatively, the device may be pre-assembled. According to some embodiments, an external pressure may be applied to initiate fluid sample flow from the first compartment.

In step 404, fluid sample passes through third compartment, wherein the third compartment comprises one or more filters configured to selectively isolate and/or preserve, and/or enable growth of matters of interest. The remaining fluid may then optionally reach a fifth (waste and/or post-filtered fluids) compartment or be otherwise expelled.

In step 406, which is an optional step according to some embodiments, washing solution passes through third compartment, allowing washing out of the fluid sample. According to some embodiments, fluid sample may include a urine sample, thereby washing out the urine after the isolating and capturing of matters of interest of step 404 (e.g. epithelial cells and exosomes found in the urine). After having passed of the one or more filters, the washing solution may likewise reach the fifth compartment, or a designated sub-section in the fifth compartment, or be expelled. According to some embodiments the fifth compartment may be or include a collection chamber configured to collect the fluid sample (the post filtered sample).

In step 408, growth solution flows into third compartment for enhancing viability and/or growth of matters of interest. During this step the fluid flow into the fifth (waste and/or post-filtered fluids) compartment is discontinued, for example, but not limited to by the closing of a one-way valve, thereby retaining the growth solution within the third compartment.

In step 410, which is an optional step according to some embodiments, accumulating and retaining of post-filtered fluids and waste occurs in fifth container. After passing through third compartment, post-filtered fluids, such as post-filtered fluid sample and/or post-filtered washing solution, are received and retained inside the fifth compartment. Optionally, the fifth compartment may be divided into one or more sub-sections, configured to separate the post-filtered fluids, for example, preventing mixing of the post-filtered fluid sample and the post-filtered washing solution. Optionally, post-filtered fluids may be dissembled for the second part of the device and transferred for a further analysis in a medical facility.

According to some embodiments, fluids flow from each of the compartment in the first part (e.g., first, second and/or fourth compartment) may be performed manually and/or automatically. While other mechanisms may also be envisaged, as elaborated herein, second and fourth compartments may include puncturing needles, configured to puncture barriers positioned in the second and the fourth compartments, separating second and fourth compartment from first compartment. This may advantageously allow a timed (i.e., sequential) flow from each of first, second and fourth compartments through one-way valve and into the third compartment containing one or more filters.

Optionally, third compartment and/or one or more filters of third compartment may be removed from selective isolation and/or growth device for further analysis (step not shown).

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1

Six prototypes of devices were fabricated and used for selectively isolating t24 bladder cancer cells with an average diameter of about 23.3 μm from a fluid sample, in accordance with the disclosure described herein. Specifically, the isolating was based on microfluidics mechanism, wherein the isolating was performed by three filters based on ridges positioned inside each of the devices. The influence of ridges sizes on the efficacy of the isolating was studied. Further, was studied the effect of flow rates of the fluid sample on the efficacy of the isolating of the cells.

The devices were 3D-printed using a Formlabs printer with SLA technology, and a BioMed Amber resin by Formlabs. Each of the devices included three 3D-printed ridges-based filters. The ridges-based filters are removable and may be further fitted into standard 6/12/24 cell growth plates, such as well plates. Channel (corresponding to channel 136) height of all the fabricated devices was about 1 mm. The approximate dimensions of the ridges (i.e. length, height and width) are listed in Table 1.

To evaluate the selective isolation and capture efficiency of the devices, the concentrations of the fluid samples were measured before and after passing of the fluid sample through the devices. The flow rates of the fluids were pre-determined and were controlled by a syringe pump (see Table 1).

In accordance with the current disclosure, upon passing of the fluid sample, the filters were washed with about 20 mL phosphate buffered saline (PBS). The drained outcome was collected and measured for identification of t24 cells. The flow rates of the fluids were pre-determined and were controlled by a syringe pump (see Table 1). Further, each of the three filters were disassociated from the device and transferred into well plates and were observed in a light microscope. The experimental parameters and the efficacy values are summarized in Table 1.

TABLE 1
Summary of the experimental parameters (ridge dimensions,
fluid sample input parameters) and the efficacy results.

| | | | | | t24 | | |
|---|---|---|---|---|---|---|---|
| | Ridges Dimensions | | | Flow | Input | t24 | Isolating |
| | Height | Length | Width | Rate | [10⁶ | Input | Efficacy |
| Device | [μm] | [mm] | [μm] | [ml/min] | cells/ml] | [ml] | [%] |
| 1 | 300 | 1.0 | 826 | 1 | 0.10 | 19.5 | 100.00 |
| 2 | 250 | 1.5 | 826 | 1 | 0.14 | 16.0 | 90.23 |
| 3 | 300 | 1.0 | 826 | 1 | 0.10 | 19.5 | 78.85 |
| 4 | 350 | 1.0 | 826 | 10 | 0.10 | 16.0 | 69.67 |
| 5 | 200 | 1.0 | 826 | 10 | 0.10 | 19.5 | 77.11 |
| 6 | 350 | 1.0 | 826 | 20 | 0.10 | 19.5 | 74.41 |

According to Table 1, it is evident that the fabricated devices exhibited satisfactory isolating efficacy values in the range of 70-100%. Efficacy of 100% was achieved by infusing the fluid sample into the first device at 1 ml/min. Increasing the flow rate by a factor of 10 and even by 20 still yielded efficacy of about 80%. Thus, demonstrating satisfactory efficacy values even at short isolating durations-less than 2 minutes.

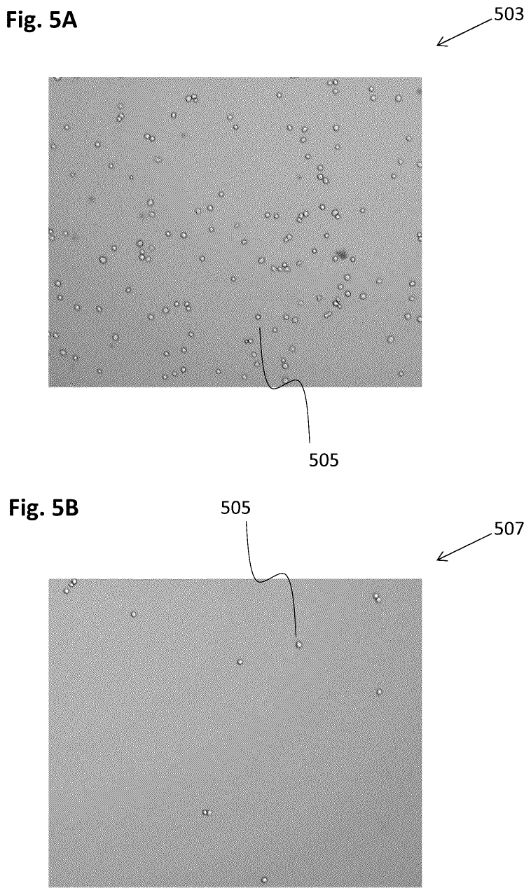

FIG. 5A shows an example of a bright-field light microscope image of a fluid sample 503 comprising t24 bladder cancer cells 505 (i.e. matters of interest) before loading into one of the experimental devices, fabricated according to some of the disclosed embodiments. FIG. 5B shows a bright-field light microscope image of an outflow (post-filtered fluid) 507 collected after passing through the one of the experimental devices. It is evident that the concentration of t24 cells 505 significantly decreased after passing through the first device.

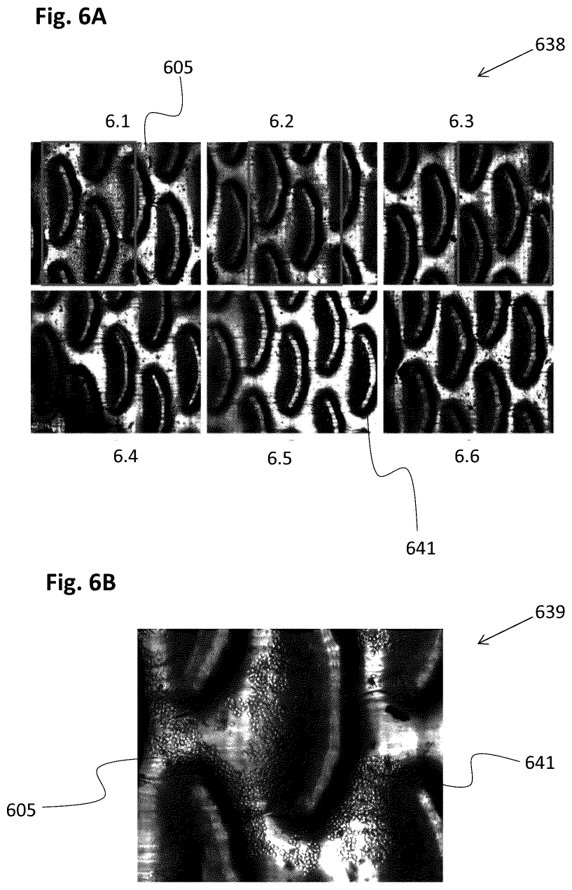

FIG. 6A shows two ridges-based filters 638 of the first device. Referring to panels 6.1-6.6, panels 6.1-6.4 demonstrate bright-field light microscope images of different areas of the first filter, such that panel 6.1 was observed from an inlet position of the first filter, while panel 6.4 was observed near an outlet of the first filter, and panels 6.5 and 6.6 demonstrate bright-field light microscope images of an inlet and outlet positions of the second filter, respectively. As previously described, each filter included ridges 641 with approximate pre-determined sizes (see Table 1). According to FIG. 6, it is evident that the concentration of the captured cells gradually decreased within the device. High concentration of cells was observed in the vicinity of the first filter inlet position (panel 6.1), then, at further locations from the first filter inlet, the observed concentration of cells gradually decreased until extremely low concentrations of cells were identified in the second filter (panels 6.5 and 6.6).

FIG. 6B shows a bright field light microscope image of captured t24 bladder cancer cells 605 captured in ridges 641 of filters of the first device. FIG. 6B demonstrates the accumulation of the captured t25 bladder cancer cells 605 in the vicinity of the ridges 641. Thus, it is understood that the captured t24 bladder cancer cells 605 may be further provided a growth solution for facilitating their preservation and/or growth.

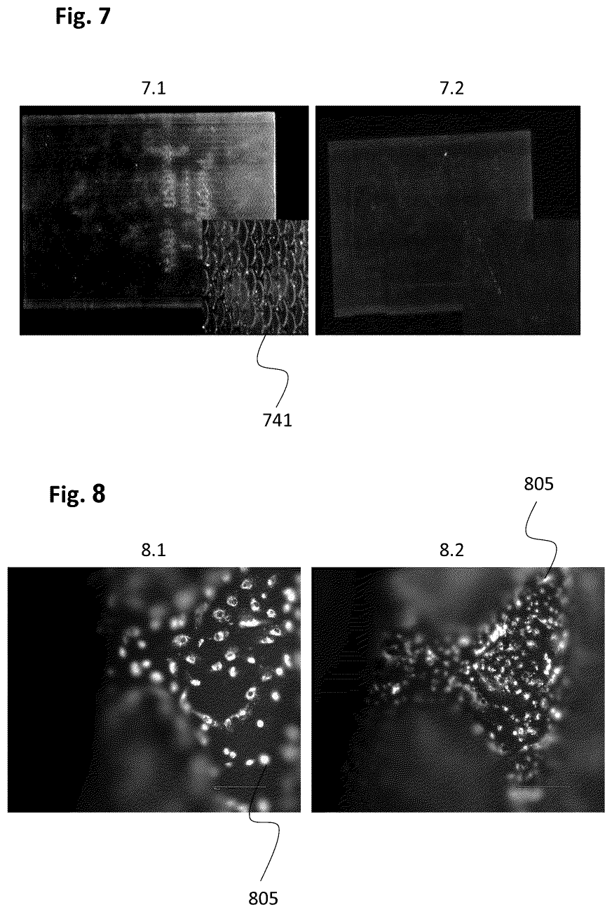

FIG. 7 shows the capacity of a filtering layer according 741 to some embodiments of the present disclose (shown in FIG. 7.1), compared to the capacity of a control filtering layer (shown in FIG. 7.2). It is evident that that the disclosed herein filtering layer 741 captured significantly higher concentration of t24-GFP cells (shown as fluorescent dots captured/trapped on the filtering layer) than the control filtering layer.

FIG. 8 shows live and healthy urine cells, whose cellular linage may be of variable origins, 805, which were stained with a cytopainter, and captured/trapped in a filtering layer of a disclosed herein device, according to some embodiments.

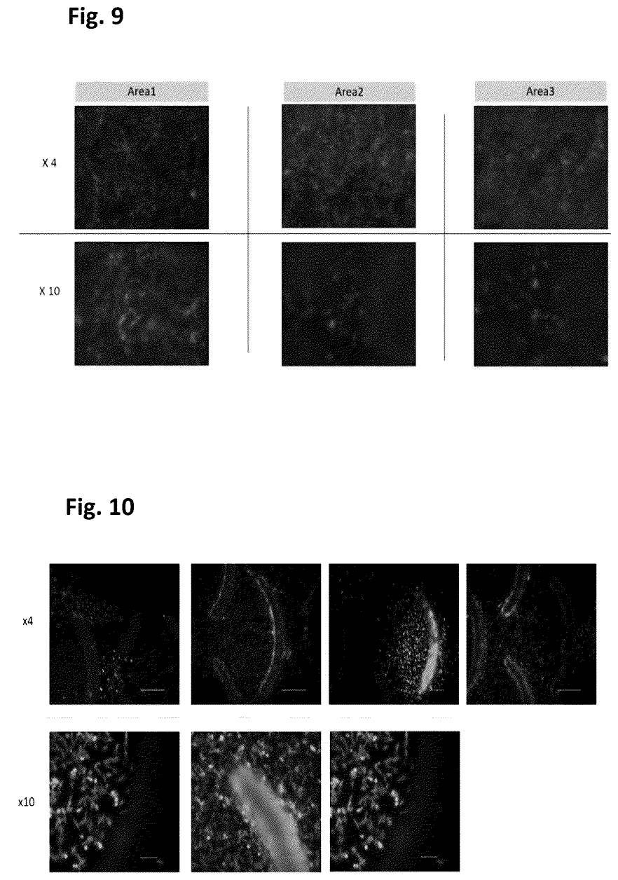

FIG. 9 shows specific capturing of a low concentration of live and healthy urine epithelial cells, captured/trapped within the filtering layer of a disclosed herein device, according to some embodiments. These urine epithelial cells, originating in the epithelium of the urinary track, were counted and diluted to a concentration of 3000 cells per 100 ml of urine and subsequently stained with a red cytopainter and a blue nuclear stain, according to some embodiments. As shown in FIG. 9, the sensitivity of the disclosed herein device, according to some embodiments, is of at least 3000 epithelial cells in 100 ml of urine.

FIG. 10 shows the compatibility for capturing/trapping of additional cell types, such as cells for cancer diagnosis. During the experiment, 3000 cancer cells of a relevant urine bladder cancer origin, marked with GFP (t25-GFP) were added to 100 ml of urine and were administered into a disclosed herein device, according to some embodiments. A few days afterwards, FIG. 10 was taken, showing the presence of cancer cells therein, thereby indicating the sensitivity of the disclosed device to capture the cancer cells therein.

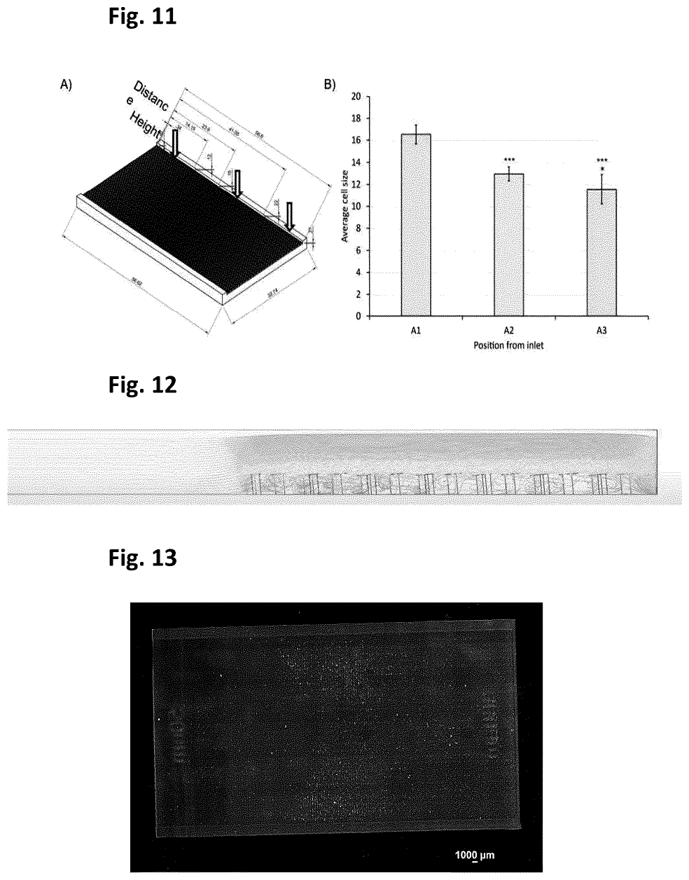

FIG. 11 shows indications of size-selective separation of a device, according to some embodiments. As shown in FIG. 11, cells having a larger average size were captured close to the inlet of the device, while cells having a smaller average size were captured further away from the inlet in a capturing device where (H) as depicted in the side views of FIGS. 3.1b-3.4b), varied along the length of the capturing device using the trapping grid depicted in FIG. 13.

FIG. 12 shows a flow simulation plot depicting liquid with cells flowing from left to right though a disclosed herein device, according to some embodiments. As shown in FIG. 12, once the cells reach the capturing/trapping zone (i.e., one or more filters), the flow changes from laminar flow to turbulent flow, due to the micro-structures (e.g., ridges) present on the one or more filters. According to some embodiments, the micro-structures are configured to push the cells flowing therethrough, thereby facilitating capturing the cells within the one or more filters of the device.

FIG. 13 shows an example of a trapping grid of one or more filters, according to some embodiments. According to some embodiments, the trapping grid is configured to capture various matters of internet having different sizes. It is understood by those skilled in the art that other types of cells and/or matters of interest may be selectively isolated by the disclosed devices. Further, it is understood that any fluid-related property (e.g., types of fluids, concentrations, volumes, viscosity), as well as any of the isolating properties (e.g., number of filters, types of filters, isolating mechanisms, flow rates, channel sizes) may vary depending on the desired application and outcome of the device. Each possibility is a separate embodiment.

1.-31. (canceled)

32. A device for a selective isolation and/or growing of matters of interest from a biological source fluid sample comprising two parts, comprising a plurality of compartments:
a first part comprising:
a first compartment configured to collect the fluid sample, and
a second compartment comprising a growth solution configured to preserve and/or facilitate growth of the matters of interest, and
a second part comprising a third compartment, wherein the third compartment comprises one or more filters configured to capture the matters of interest;
wherein the first compartment comprises a one-way valve which when open allows flow of the fluid sample from the first compartment into the third compartment through the one or more filters, and wherein the second compartment configured to allow flow of the growth solution from the second compartment into the third compartment, such that the matters of interest captured by the one or more filters are covered by the growth solution.

33. The device of claim 32, further comprising a fourth compartment, comprising a washing solution for washing the one or more filters before and/or after the selective isolating of the matters of interest.

34. The device of claim 32, further comprising a fifth compartment, configured to accumulate post-filtered fluids, such as a post-filtered fluid sample after passing through the third compartment.

35. The device of claim 34, wherein the fifth compartment comprises one or more separate sub-sections, configured to separate the post-filtered fluid sample and the washing solution after passing through the third compartment.

36. The device of claim 34, further comprising a second one-way valve which in its open configuration is configured to allow flow from the third compartment to the fifth compartment and in its closed configuration is configured to prevent flow from the third compartment to the fifth compartment, wherein the second one-way valve is normally open and configured to assume its closed configuration with initiation of flow of the growth solution from the second compartment into the third compartment, thereby retaining the growth solution within the third compartment.

37. The device of claim 32, wherein the one or more filters comprise an inertial microfluid fractionation element configured to selectively isolate the matters of interest from the fluid sample, wherein the inertial microfluid fractionation element a plurality of microscopic and/or nanoscopic ridges of variable sizes configured to selectively isolate the matters of interest according to their size and/or weight.

38. The device of claim 32, wherein the one or more filters comprise one or more micro and/or nano filters configured to selectively isolate the matters of interest from the fluid sample according to their size and/or weight.

39. The device of claim 32, wherein at least a wall of the first compartment is sufficiently flexible to allow compression of the first compartment when external pressure is applied thereon, wherein the one-way valve is configured to assume its open configuration with compression of the first compartment.

40. The device of claim 32, further comprising one or a plurality of puncturing needles configured to initialize flow of the growth solution from the second compartment into the third compartment.

41. The device of claim 32, further comprising a plurality of sensors for monitoring one or more parameters associated with the quality performance of the device.

42. The device of claim 32, wherein the one or more filters further comprise electrically conducting element configured to capture the matter of interest based on electrostatic forces, affinity, and/or ion exchange procedures of the fluid sample components.

43. The device of claim 32, further comprising one or more vacuum pumps configured to initialize flow of the growth solution from the second compartment to the third compartment.

44. The device of claim 32, further comprising one or more Bernoulli pumps configured to initialize flow of the growth solution from the second compartment to the third compartment.

45. The device of claim 32, further comprising one or a plurality of sensors for monitoring transport-related parameters, including temperature and/or angular velocity parameters.

46. The device of claim 32, further comprising one or a plurality of sensors for identification of presence of the matters of interest.

47. The device of claim 32, wherein the fluid is a urine sample.

48. The device of claim 32, wherein the matters of interest are epithelial cells and/or exosomes, and/or extracellular vesicles, and/or apoptotic bodies, and/or cell free RNA DNA, and/or proteins, and/or cancerous cells.

49. A method for a point-of-care selective isolating and/or growing of matters of interest from a biological source fluid sample comprising:
providing a device comprising:
a first part, comprising a first compartment comprising the fluid sample and a one-way valve in a closed configuration, and
a second compartment comprising a growth solution
a second part, comprising a third compartment comprising one or more filters;
collecting the fluid sample into the first compartment;
shifting the first one-way valve to an open configuration;
passing of the fluid sample through the third compartment, such that the fluid sample passes through the one or more filters, thereby capturing the matters of interest;
passing of the growth solution into the third compartment, wherein the growth solution preserves and/or facilitates growth of the matters of interest.

50. The method of claim 49, wherein a source of the fluid sample is a subject with a neurological disorder, mental and/or physical disability, and/or cancer.

51. The device of claim 32, wherein the fluid is a urine sample.
